(19) United States
(12) Patent Application Publication
(10) Pub. No.: US 2009/0112171 A1
(43) Pub. Date: Apr. 30, 2009

(54) Urine bag pocket

(75) Inventors: Frency Sau Fun Ng, Kowloon (CN); Patrick Chi-leung Hui, Kowloon (CN)

Correspondence Address: FRENCY SAU FUN NG, THE HONG KONG POLYTECHNIC UNIVERSITY, HUNG HOM, KOWLOON, HK

(73) Assignee: The Hong Kong Polytechnic University, Kowloon (HK)

(21) Appl. No.: 11/976,777

(22) Filed: Oct. 29, 2007

(51) Int. Cl. A61F 5/44; A61M 1/00

(52) U.S. Cl. 604/332; 604/327

(57) ABSTRACT

The present invention relates to clothing items such as pants or shoulder bags incorporating urine bag pockets suitable for holding a urine bag. Through the invention, incontinence sufferers are able to maintain mobility while still concealing a urine bag.

### BACKGROUND

Incontinence refers to a patient's inability to control the evacuation of the bladder. The condition can be caused by a number of reasons, such as stress, strong urge to urinate, overflow, age, or transient. To address incontinence and allow a patient mobility, urinary catheterization is utilized. The urine delivered via the catheter is usually collected in a urine bag.

Many patients who wear a urine bag do not like the unusual appearance. The appearance of carrying a urine bag hurts a patient's confidence and self-esteem. Most patients prefer to conceal the urine bag in the best way, for example, hanging the urine bag on their waist and covering it up by an overcoat, or tying the urine bag on the leg and covering it with pants.

Further, the urine bag is required to be emptied. This is performed via valve. This valve is required to be readily accessible, while hidden at the same time.

It is an object of the present invention to provide an improvement over prior art devices for camouflaging urine bags.

### DESCRIPTION

The present invention relates to clothing incorporating a urine bag pocket suitable for holding a urine bag. Clothing items include pants, shorts, coats, bags, etc. The urine bag pocket can be positioned anywhere on the clothing item, provided the catheter is allowed an easy path to the penis or bladder. Through the present invention, incontinence sufferers maintain dignity while still being mobile as the urine bag is sufficiently camouflaged.

These and other features, aspects, and advantages of the apparatus and methods of the present invention will become better understood from the following description, appended claims, and accompanying drawings where:

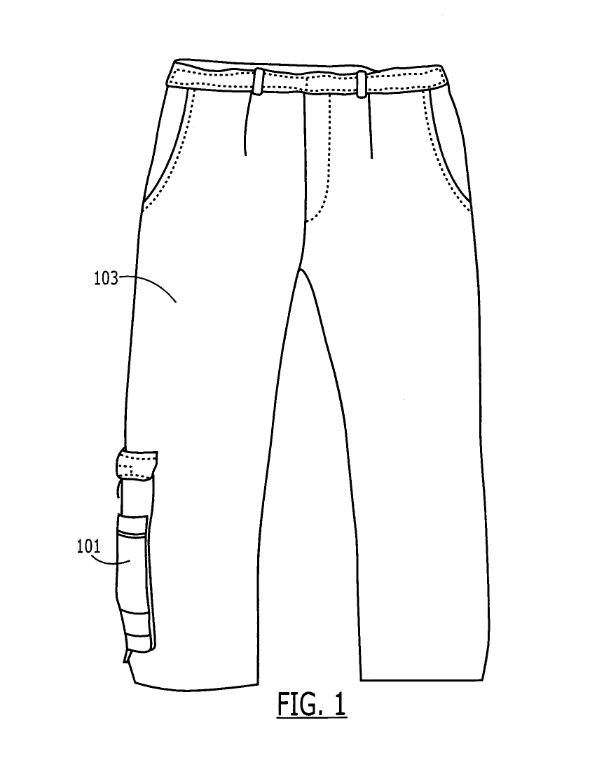

FIG. 1 shows an embodiment of a clothing item incorporating the present urine bag pocket;

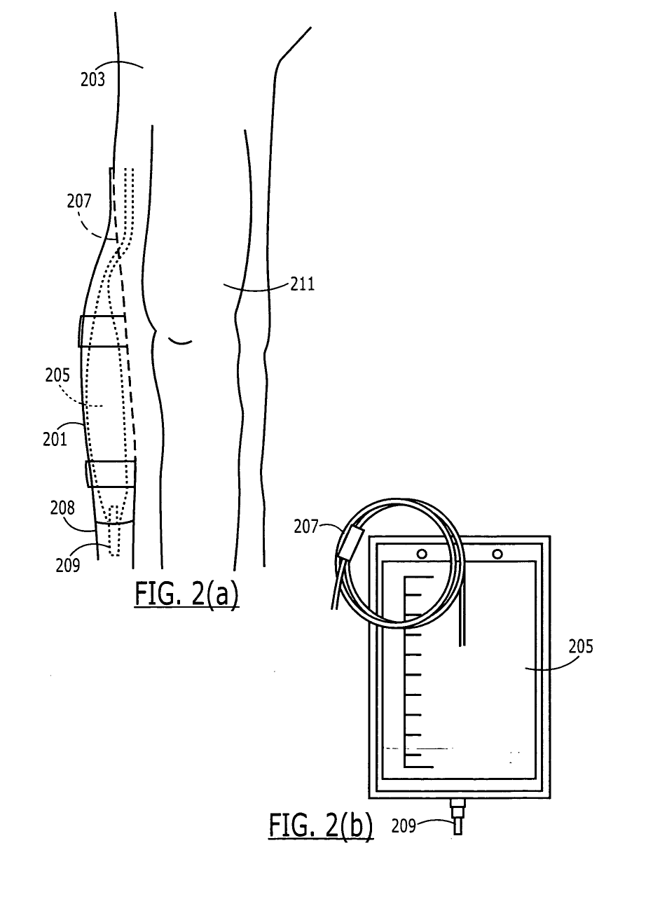

FIG. 2(a) shows the urine bag pocket in a clothing item is relation to a patient's leg;

FIG. 2(b) shows a urine bag to be inserted into the present urine bag pocket;

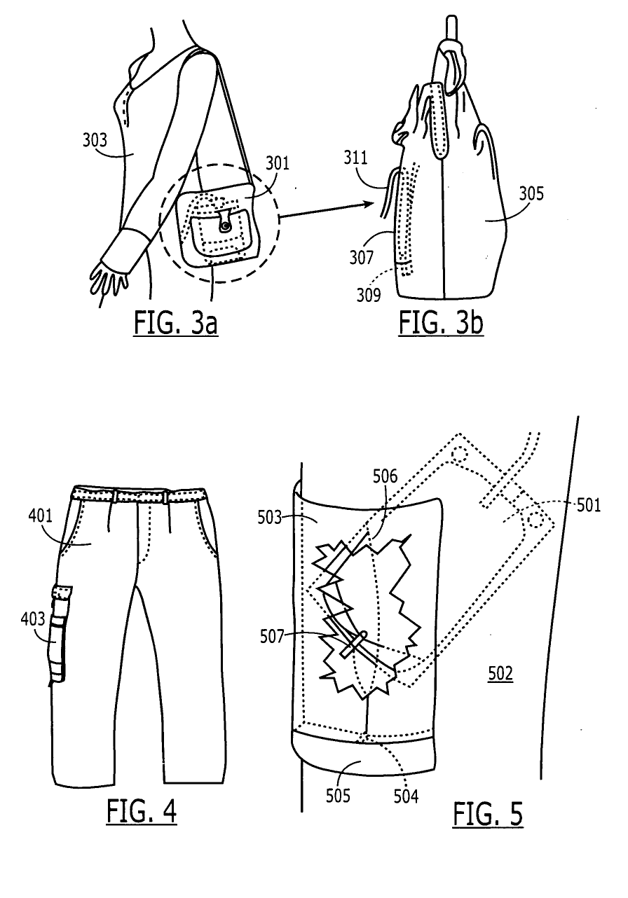

FIG. 3 shows a bag incorporating the present urine bag pocket;

FIG. 4 shows the present urine bag pocket in a different position on the clothing item; and

FIG. 5 shows the insertion of a urine bag into the urine bag pocket.

The following description of certain exemplary embodiment(s) is merely exemplary in nature and is in no way intended to limit the invention, its application, or uses.

Now, to FIGS. 1-5,

FIG. 1 is an embodiment of a pants 103 incorporating the urine bag pocket 101. In this particular embodiment, the urine bag pocket 101 is located near the lower portion of one pant leg. The bag pocket 101 is positioned snug against the clothing item 101 and on the outside. The size of pocket 101 can vary, dependent on the size of the urine bag to be incorporated in the pocket 101. The appearance of the pocket 101 preferably matches the clothing item 103, such as having the same color, texture, etc.

FIG. 2(a) shows the incorporation of the urine bag 205 in the urine bag pocket 201 of a clothing item 203.

As shown, the urine bag pocket 201 is of a dimension to match the urine bag 203. The pocket 201 possesses an opening flap (not shown) on its backside whereby the urine bag 205 is inserted. The pocket 201 also possesses a downward flap 208. The downward flap 208 helps to further camouflage the bag 205 while still allowing easy access to the discharge valve 209, from which the bag 205 can be emptied of urine. The pocket 201 also includes an input for the catheter tube 207 from the user's penis or bladder. The catheter tube 207 can be directed against the user's leg 211. In an alternative embodiment, the catheter tube 207 can be directed along a channel on the inside of the clothing item 203, for example a seam.

FIG. 2(b) shows a urine bag 205 suitable for placement in a clothing item of the present invention. The urine bag 205 includes a catheter 207 and tube for insertion into a user's penis or bladder, and a discharge valve 209 for emptying the bag 205.

FIG. 3(a) shows an embodiment of a clothing item incorporating the urine bag pocket 301, wherein the clothing item is a shoulder bag 301. In use, the shoulder bag 301 is carried adjacent to the waist of the user, 303.

As shown in FIG. 3(b), the urine bag pocket 307 is positioned at the back side of the shoulder 305. The urine bag packet 307 possesses an outlet whereby the catheter tube can exit from, as well as a cover flap 309 for emptying the urine bag. As stated, the shoulder bag 305 is carried adjacent to the user's waist. The catheter tube 311 is directed to the penis or bladder of the user. The way the shoulder bag 305 is carried, the catheter tube 311 is hidden.

FIG. 4 is a further embodiment of the present invention. In this embodiment, the urine bag pocket 403 is positioned on the upper side of a pair of pants 401.

FIG. 5 shows the insertion of a urine bag 501 into the present urine bag pocket 503. The urine bag pocket 503 possesses an opening 506 on its backside into which the urine bag 501 is inserted. The opening 506 can be closed with, for example, a zipper, hook-and-loop, or fasteners. After insertion, the discharge valve 507 of the urine bag 501 is inserted through a lower opening 504. A downward flap 505 covers the discharge valve 507 after it is inserted through the lower opening 504.

Having described embodiments of the present system with reference to the accompanying drawings, it is to be understood that the present system is not limited to the precise embodiments, and that various changes and modifications may be effected therein by one having ordinary skill in the art without departing from the scope or spirit as defined in the appended claims.

In interpreting the appended claims, it should be understood that:

a) the word “comprising” does not exclude the presence of other elements or acts than those listed in the given claim;

b) the word “a” or “an” preceding an element does not exclude the presence of a plurality of such elements;

c) any reference signs in the claims do not limit their scope;

d) any of the disclosed devices or portions thereof may be combined together or separated into further portions unless specifically stated otherwise; and

e) no specific sequence of acts or steps is intended to be required unless specifically indicated.

1. A clothing item having a urine bag pocket, attached thereto, wherein said urine bag pocket comprises;
an opening on the back of said urine bag pocket having a sealable device;
a lower opening for accepting a discharge valve of a urine bag; and
a downward flap for concealing said discharge valve;
wherein said urine bag pocket is permanently attached to said clothing item.

2. The clothing item of claim 1, wherein said clothing item can be selected from the group consisting of a pair of pants, a coat, a skirt, and a shoulder bag.

3. The clothing item of claim 1, wherein said sealable device is selected from the group consisting of a zipper, hook-and-loop, and fasteners.

4. The clothing item of claim 1, wherein said urine bag pocket is positioned near the lower extremity of a pair of pants.

5. A urine bag pocket, comprising
a urine bag having a bag, a catheter tube, and a discharge valve;
a pocket having an opening on its backside having a sealable device, a lower opening for accepting said discharge valve, and a downward flap for concealing said discharge.

6. The clothing item of claim 1, further comprising a urine bag having a bag, a catheter tube, and a discharge valve.
